(19) United States
(12) Patent Application Publication
(10) Pub. No.: US 2013/0030369 A1
(43) Pub. Date: Jan. 31, 2013

(54) VASCULAR INTRODUCER INCLUDING EXPANDABLE PASSAGE MEMBER

(71) Applicants: Howard Root, Minneapolis, MN (US); Gregg Sutton, Maple Grove, MN (US)

(72) Inventors: Howard Root, Minneapolis, MN (US); Gregg Sutton, Maple Grove, MN (US)

(73) Assignee: Vascular Solutions, Inc., Minneapolis, MN (US)

(21) Appl. No.: 13/191,889

(22) Filed: Jul. 27, 2011

(51) Int. Cl. A61B 17/34

(52) U.S. Cl. 604/164.03

(57) ABSTRACT

Vascular introducer systems, kits, and methods providing or creating access to vessels, such as radial or femoral arteries, are disclosed. A vascular introducer system includes a removable inner tubular member, a removable outer tubular, tear-away member, and an expandable passage member positioned between the inner and outer tubular members. The removable inner tubular member and the removable outer tubular, tear-away member help protect and maintain a contracted configuration of the expandable passage member when the introducer system is advanced into a vessel. The expandable passage member includes an inner surface configured to receive an elongate treatment device, for example, following removal of the inner and outer tubular members. In some examples, the expandable passage member includes one or more kink-resistant members extending along a portion of the passage member. In various examples, the expandable passage member includes a wall thickness sufficient to protect vessel surfaces, while preserving vessel access size.

### TECHNICAL FIELD

This patent document pertains generally to systems, kits, and methods to provide or create vessel access. More particularly, but not by way of limitation, this patent document pertains to vascular introducer systems, kits, and methods including an expandable passage member configured to protect vessel surfaces, while preserving vessel access size.

### BACKGROUND

Minimally invasive procedures have been implemented in a variety of medical settings, such as for vascular interventions, stenting, embolic protection, electrical heart stimulation, heart mapping and visualization, and the like. These procedures generally rely on accurately navigating and placing treatment devices within a subject's vasculature.

During minimally invasive procedures, a target vessel can be accessed through a small access hole. The small access hole is usually initiated by piercing the skin, the target vessel, and any intermediate body structures using a needle (e.g., a trocar). With the needle in place, a guide wire can be advanced within an inner lumen of the needle and into the target vessel, thereby providing a “railway” to the vessel. Upon removing the needle by sliding it off a proximal end of the railway, one or more elongate treatment devices (e.g., diagnostic catheters, electrical leads, and other interventional devices) can be advanced over the guide wire and into the vessel. Thus, a diagnostic or therapeutic procedure can be performed by advancing one or more treatment devices over this railway.

There are many risks involved with advancing treatment devices over a guide wire and into a vessel. For example, a treatment device can skive or otherwise damage a wall of the vessel, particularly as the device is introduced into the vessel or passes through narrow passages or tortuous vessel anatomy involving sharp bends. Advancement of treatment devices also risks dislodging embolic material or even perforating the vessel wall.

### OVERVIEW

To help minimize or prevent damage to a vessel wall during insertion and removal of a treatment device, a fixed-diameter tubular introducer sheath is often used by caregivers to act as an intermediary between an outer surface of the treatment device and the vessel wall. However, conventional tubular introducer sheaths have relatively large cross-sectional sizes. These large cross-sections make it difficult, if not impossible, to internally advance treatment devices having an outer diametrical size greater than an effective vessel diameter (i.e., a vessel's natural diameter downsized to account for the space occupied by the tubular introducer sheath). Accordingly, many minimally invasive procedures that would desirably be performed by a caregiver using a radial artery are rerouted to a larger femoral artery. Similarly, other minimally invasive procedures that would desirably be performed by caregivers using a femoral artery are rerouted elsewhere.

The present inventors recognize, among other things, a need for gaining access into a vessel of a subject, such as a radial or femoral artery, while protecting vessel walls and preserving vessel access size (e.g., effective vessel diameter or cross-sectional area). Using this larger-than-conventional access size, one or more elongate treatment devices can be efficiently introduced into a desired vessel during a minimally invasive procedure.

The present vascular introducer systems, kits, and methods are configured to provide or create access to vessels. A vascular introducer system includes a removable inner tubular member, a removable outer tubular, tear-away member (e.g., a peel-away member), and an expandable passage member having low column strength positioned between the inner and outer tubular members. The removable inner tubular member and the removable outer tubular, tear-away member can help protect (e.g., prevent “bunching”) and maintain a contracted configuration of the expandable passage member when the vascular introducer system is advanced into a vessel. The expandable passage member can include an inner surface forming an introduction channel configured to receive an elongate treatment device, such as following removal of both the inner and outer tubular members. In some examples, the expandable passage member includes one or more kink-resistant members extending along a length portion of the passage member. In various examples, the expandable passage member includes a wall thickness sufficient to protect vessel walls, while preserving vessel access size.

To better illustrate the vascular introducer systems, kits, and methods disclosed herein, a non-limiting list of examples is provided here:

In Example 1, a vascular introducer system comprises a removable inner tubular member; a removable outer tubular, tear-away member; and an expandable passage member positioned between the inner tubular member and the outer tubular, tear-away member. The expandable passage member is configured to sufficiently allow for controlled inelastic radial expansion upon the application of a radial force thereto. An inner surface of the expandable passage member is configured to receive an elongate treatment device following removal of both the inner and the outer tubular members.

In Example 2, the vascular introducer system of Example 1 is optionally configured such that the expandable passage member includes a folded, wrapped, or rolled polymer member extending about a circumference of the inner tubular member.

In Example 3, the vascular introducer system of any one or any combination of Examples 1 or 2 is optionally configured such that the expandable passage member includes a wall thickness of about 0.001 inches to about 0.002 inches.

In Example 4, the vascular introducer system of any one or any combination of Examples 1-3 is optionally configured such that an inner diameter of the outer tubular, tear-away member is less than an inner diameter of the expandable passage member, post-expansion.

In Example 5, the vascular introducer system of any one or any combination of Examples 1-4 is optionally configured such that the outer tubular, tear-away member includes an outer surface diameter of about 6-Fr or less.

In Example 6, the vascular introducer system of any one or any combination of Examples 1-5 is optionally configured such that an initial configuration of the expandable passage member is maintained during implantation within the vessel by the outer tubular, tear-away member and by the inner tubular member.

In Example 7, the vascular introducer system of any one or any combination of Examples 1-6 is optionally configured such that the expandable passage member includes one or more kink-resistant members extending along a length portion of the expandable passage member.

In Example 8, the vascular introducer system of Example 7 is optionally configured such that the one or more kink-resistant members extend along one side of the expandable passage member.

In Example 9, the vascular introducer system of any one or any combination of Examples 7 or 8 is optionally configured such that the one or more kink-resistant members include a wire configuration having an outer diameter of about 0.004 inches or less.

In Example 10, the vascular introducer system of any one or any combination of Examples 7-9 is optionally configured such that the one or more kink-resistant members are embedded within, or attached to, a wall of the expandable passage member.

In Example 11, the vascular introducer system of any one or any combination of Examples 7-9 is optionally configured such that the expandable passage member includes an inner passage member and an outer passage member, and the one or more kink-resistant members are positioned between a wall of the inner passage member and a wall of the outer passage member.

In Example 12, the vascular introducer system of any one or any combination of Examples 1-11 is optionally configured such that there is a lack of bonding attachment between the inner tubular member and the expandable passage member and between the outer tubular, tear-away member and the expandable passage member.

In Example 13, the vascular introducer system of any one or any combination of Examples 1-12 is optionally configured such that the expandable passage member includes an outer surface configured to contact an inner surface of a vessel following removal of the outer tubular, tear-away member.

In Example 14, a kit comprises a needle; a guide wire; the vascular introducer system of any one or any combination of Examples 1-13; and instructions for using the vascular introducer system to insert an elongate treatment device into a radial or a femoral artery.

In Example 15, a method comprises inserting at least a portion of an inner tubular member, an expandable passage member, and an outer tubular member into a vessel; separating and removing the outer tubular member; removing the inner tubular member; and radially expanding an inner surface of the expandable passage member from a first diametrical size to a larger, second diametrical size, including introducing a radial force against the inner surface sufficient to inelastically expand the expandable passage member.

In Example 16, the method of Example 15 is optionally configured such that introducing the radial force includes inserting an elongate treatment device, having an outer diameter greater than an outer diameter of the inner tubular member, into the expandable passage member thereby radially expanding the expandable passage member from a proximal end portion to a distal end portion.

In Example 17, the method of Example 16 is optionally configured such that introducing the elongate treatment device into the expandable passage member includes protecting the vessel by inhibiting direct contact between an inner vessel surface and an outer surface of the elongate treatment device.

In Example 18, the method of any one or any combination of Examples 15-17 is optionally configured such that introducing the radial force includes introducing an elongate treatment device, having an outer diameter greater than about 6-Fr, into a radial artery such that a wall of the expandable passage member is positioned intermediate an outer surface of the treatment device and an inner surface of the radial artery.

In Example 19, the method of any one or any combination of Examples 15-18 is optionally configured such that introducing the radial force includes introducing an elongate treatment device, having an outer diameter greater than about 9-Fr, into a femoral artery such that a wall of the expandable passage member is positioned intermediate an outer surface of the treatment device and an inner surface of the femoral artery.

In Example 20, the method of any one or any combination of Examples 15-19 is optionally configured such that inserting the inner tubular member, the expandable passage member, and the outer tubular member into the vessel includes guiding an inner lumen of the inner tubular member over a guide wire.

In Example 21, the method of any one or any combination of Examples 15-20 is optionally configured such that inserting the inner tubular member, the expandable passage member, and the outer tubular member into the vessel includes inserting the members into a radial artery.

In Example 22, the method of any one or any combination of Examples 15-20 is optionally configured such that inserting the inner tubular member, the expandable passage member, and the outer tubular member into the vessel includes inserting the members into a femoral artery.

In Example 23, the method of any one or any combination of Examples 15-22 is optionally configured such that radially expanding the inner surface of the expandable passage member includes increasing the diametrical size of the inner surface by at least about 100%.

In Example 24, the system, kit, or method of any one or any combination of Examples 1-23 is optionally configured such that all elements or options recited are available to use or select from.

These and other examples and features of the present vascular introducer systems, kits, and methods will be set forth in part in following Detailed Description. This Overview is intended to provide non-limiting examples of the present subject matter—it is not intended to provide an exclusive or exhaustive explanation. The Detailed Description below is included to provide further information about the present vascular introducer systems, kits, and methods.

### BRIEF DESCRIPTION OF THE DRAWINGS

In the drawings, like numerals can be used to describe similar elements throughout the several views. Like numerals having different letter suffixes can be used to represent different views of similar elements. The drawings illustrate generally, by way of example, but not by way of limitation, various embodiments discussed in the present document.

FIG. 1 illustrates vascular structures providing suitable environments in which a vascular introducer system, as constructed in accordance with at least one embodiment, can be used.

FIG. 2 illustrates an isometric plan view of an example vascular introducer system, as constructed in accordance with at least one embodiment.

FIG. 3 illustrates an example method of using a vascular introducer system, as constructed in accordance with at least one embodiment.

FIG. 4A illustrates a proximal end view of an example vascular introducer system, as constructed in accordance with at least one embodiment.

FIG. 4B illustrates a side view of an example vascular introducer system, as constructed in accordance with at least one embodiment.

FIG. 5A illustrates a side, cross-sectional view of an example vascular introducer system, as constructed in accordance with at least one embodiment.

FIG. 5B illustrates a side, cross-sectional view of a distal portion of an example vascular introducer system, as constructed in accordance with at least one embodiment.

FIG. 6 illustrates a transverse cross-sectional view of an example vascular introducer system, as constructed in accordance with at least one embodiment.

FIG. 7 illustrates a side view of an example removable outer tubular, tear-away member, as constructed in accordance with at least one embodiment.

FIG. 8 illustrates a side view of an example expandable passage member, as constructed in accordance with at least one embodiment.

FIG. 9 illustrates a transverse cross-sectional view of an example expandable passage member along its length, as constructed in accordance with at least one embodiment.

FIG. 10 illustrates a side view of an example removable inner tubular member, as constructed in accordance with at least one embodiment.

### DETAILED DESCRIPTION

The present inventors recognize that it can be desirable to provide caregivers with the ability to introduce treatment devices, such as diagnostic or therapeutic devices, having an outer surface diameter approximately equal to, or in some cases greater than, the natural inner diameter of a vessel. At the same time, the present inventors recognize the importance of a low-resistance member being placed between the outer surface of the treatment device and the inner surface of a vessel wall to inhibit direct contact treatment device-vessel contact. In the absence of a low-resistance member being placed between the outer surface of the treatment device and the inner wall surface of the vessel, the vessel wall may be damaged, a subject may experience pain or discomfort as the treatment device is being introduced into the vessel (e.g., due to axial forces being imparted to the vessel tissue), and/or the vessel may involuntarily spasm, preventing internal advancement of the treatment device.

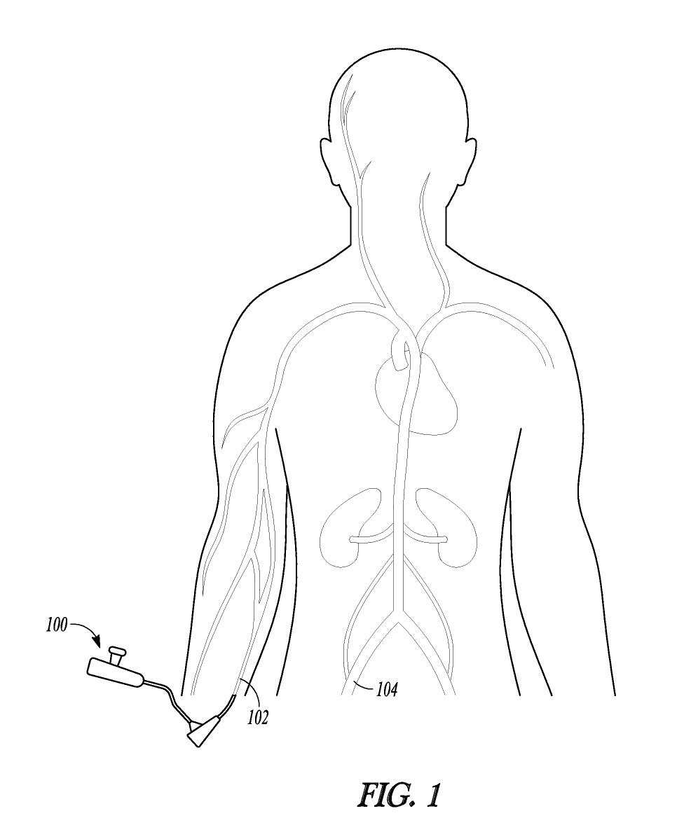

FIG. 1 illustrates vascular structures, including radial and femoral arteries, which provide suitable environments for using the present vascular introducer systems 100, kits, and methods. A radial artery 102 is located in a subject's forearm and, for a typical adult, has a natural inner diameter sufficient to allow percutaneous placement of a tubular introducer sheath having a size of 6-Fr. A femoral artery 104 is partially located in a subject's groin area and, for a typical adult, has a natural inner diameter sufficient to allow percutaneous placement of a tubular introducer sheath having a size of 9-Fr.

In certain circumstances, it can be advantageous to perform a minimally invasive procedure through the radial artery 102 rather than the larger, femoral artery 104. For example, vascular access through the radial artery 102 can help to reduce recovery time. However, typical minimally invasive procedures performed using a conventional thick, fixed-diameter tubular sheath cannot be achieved through the smaller radial artery 102, because the sheath itself occupies too much of the valuable cross-sectional access afforded by the radial artery 102. Conventional sheathless vascular introducer systems seek to solve this problem and provide the advantage of not losing part of a vessel's cross-section to a sheath. However, such sheathless systems suffer from the lack of any vessel protection during a minimally invasive procedure, such as during the introduction of a treatment device.

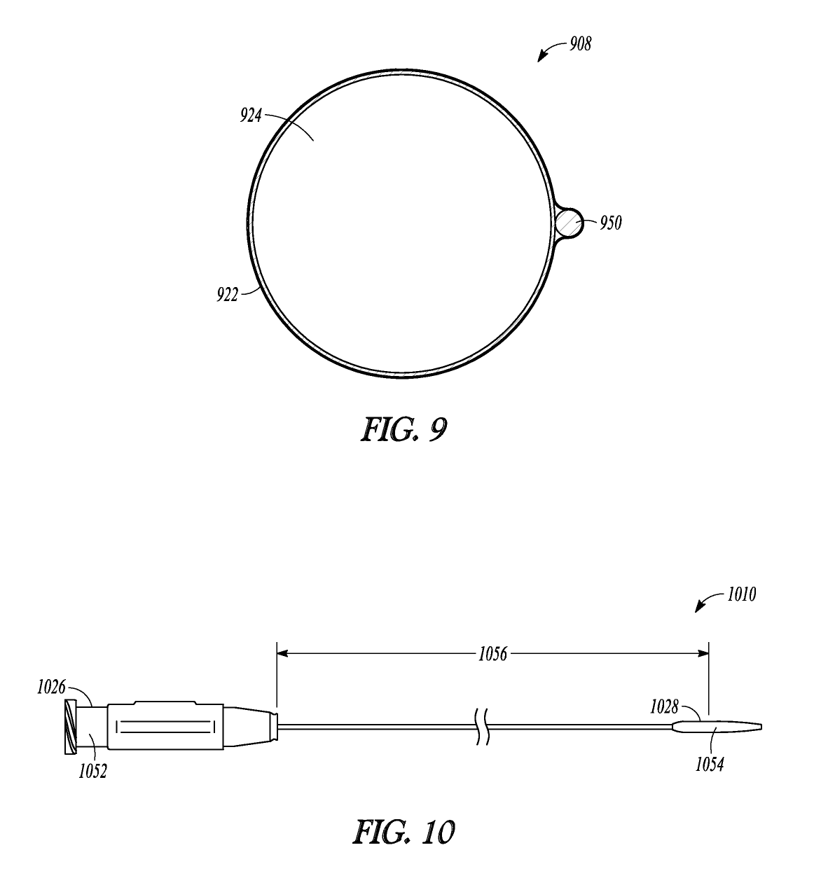

A technological concept of the present vascular introducer systems 100, kits, and methods is to provide a vessel-protecting, expandable passage that is capable of achieving an inner diameter approximately equal to or greater than a 6-Fr tubular introducer sheath for radial arteries 102 and approximately equal to or greater than a 9-Fr tubular introducer sheath for femoral arteries 104, for example, while preserving vessel cross-section through the use of a thin-walled member (see, e.g., FIG. 9). The expandable passage can be used for introducing diagnostic catheters, guide catheters, electrical leads, or other elongated treatment devices into a vessel or for draining or delivering fluids from body cavities. The expandable passage, once established within a vessel, can provide protection to the vessel by preventing direct contact between the vessel and an outer surface of the treatment device.

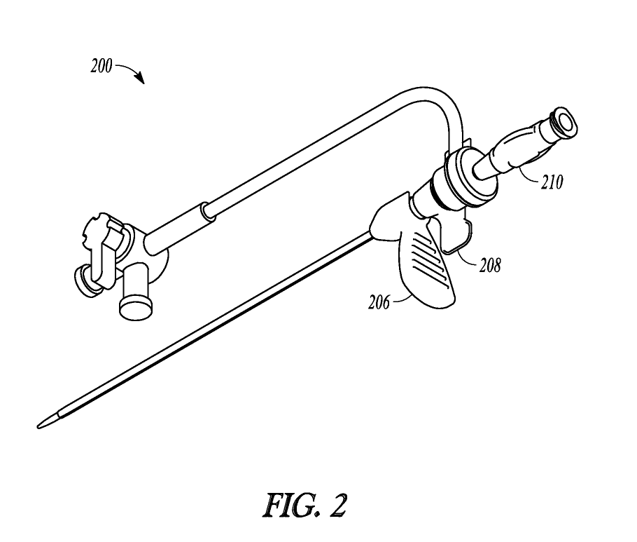

FIG. 2 illustrates an isometric plan view of an example vascular introducer system 200 providing an expandable passage. The vascular introducer system 200 can comprise a relatively rigid outer tubular, tear-away member 206, an expandable passage member 208, and an inner tubular member 210 (e.g., a dilator). The expandable passage member 208 can include a thin-walled, low column strength polymer material, which is configured to allow for controlled inelastic radial expansion upon the application of an outwardly-urging radial force. The integrity of the thin-walled, low column strength expandable passage member 208 can be preserved during vessel implantation of the introducer system 200 by the outer tubular, tear-away member 206 on the outside and by the inner tubular member 210 on the inside. Together, the outer tubular, tear-away member 206 and the inner tubular member 210 can prevent bunching or other deformation of the expandable passage member 208 during implant.

A kit can comprise the vascular introducer system 200, a needle, a guide wire, and instructions for using the vascular introducer system 200 to insert an elongate treatment device, for example, into a radial artery 102 (FIG. 1) or a femoral 104 artery (FIG. 1). The needle can provide initial access to the radial artery 102 or femoral artery 104 by piercing the skin and any intermediate body structures. With the needle in place, the guide wire can be advanced through the intermediate body structures and into the artery 102 or 104, thereby providing a “railway” to the artery. Upon removal of the needle, an inner lumen of the inner tubular member 210 can be passed over an end of the guide wire, and the vascular introducer system 200 can be advanced within the target vessel. Optionally, the kit can include additional inner tubular members 210 of various sizes to gradually urge radial expansion of the expandable passage member 208 prior to receiving an elongate treatment device.

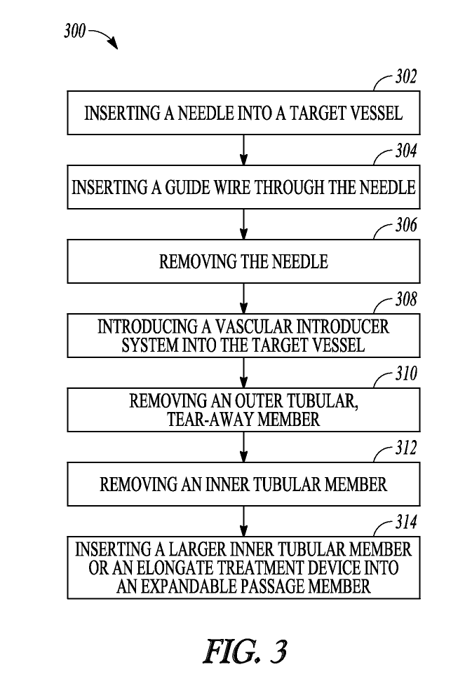

FIG. 3 illustrates an example method 300 of using a vascular introducer system as conceived by the present inventors. The vascular introducer system can be implanted by first inserting a needle into a target vessel in operation 302. As an alternative to a vessel, the target can be a hollow body organ, solid tissue location, body cavity, or the like. A guide wire can then be inserted through an inner lumen of the needle, in operation 304, and into the target vessel, thereby providing a “railway” to the vessel. Once the guide wire is in place, the needle can be removed in operation 306. The vascular introducer system can be introduced into the target vessel, in operation 308, using an over-the-guide wire technique, with the guide wire passing through an inner lumen of an inner tubular member of the vascular introducer system. The inner tubular member can include an atraumatic distal end portion that leads the way into the target vessel.

Once introduced into the target vessel, a relatively rigid outer tubular, tear-away member of the vascular introducer system can be removed, in operation 310, such as by splitting, peeling, cutting, or otherwise separating it along a preformed split, score line, linear orientation, or other structure allowing linear tearing. After the tear-away member is removed, an expandable passage member and the inner tubular member remain. Accordingly, radial expansion of the expandable passage member is no longer limited in a radially-outward direction by the relatively rigid outer tubular, tear-away member.

The inner tubular member can be removed, in operation 312, and replaced with a larger inner tubular member or an elongate treatment device in operation 314 to radially expand the passage member—because of an interference fit—along its length from a first diametrical size to a larger, second diametrical size. In an example, the inner tubular member may be caused to unfold or rotate about its axis during removal as a result of a contracted (e.g., folded or twisted) configuration of the expandable passage member. The radial force provided by the larger inner tubular member or the elongate treatment device against an inner surface of the expandable passage member can inelastically expand the wall of the passage member. An introduction channel defined by the wall provides protected access to and within the target vessel. In use, it has been found that the expandable passage member does not collapse in the absence of an inner tubular member or a treatment device, as blood flows up the introduction channel, expanding it outward under pressure against a vessel wall without leaking at the puncture of the vessel wall or anywhere along the proximal end of the passage member.

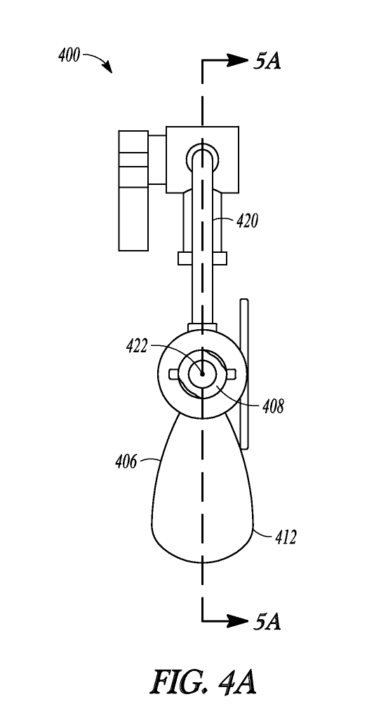

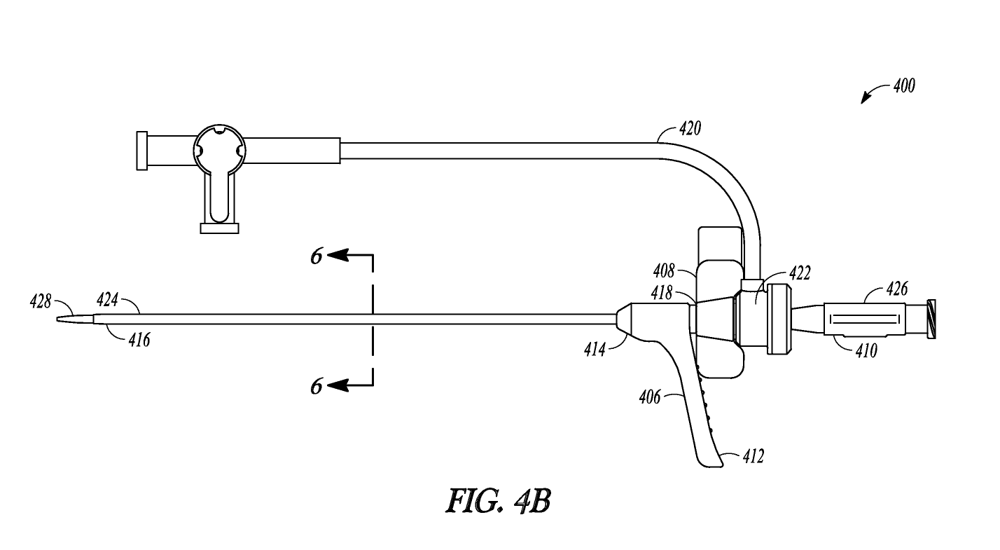

FIGS. 4A and 4B illustrate proximal end and side views of an example vascular introducer system 400. The vascular introducer system 400 can comprise a relatively rigid outer tubular, tear-away member 406, an expandable passage member 408, and an inner tubular member 410. Each of the outer tubular, tear-away member 406, the expandable passage member 408, and the inner tubular member 410 can longitudinally extend between proximal and distal ends. For example, the outer tubular, tear-away member 406 can extend from a proximal end 414, including a user-engagable peel or tear tab 412, to a distal end 416. The expandable passage member 408 can extend from a proximal end 418, connected to a side-arm member 420 and including a valve member 422, to a distal end 424. In various examples, the distal end 424 of the expandable passage member 408 terminates proximal to the distal end 416 of the outer tubular, tear-away member 406, thereby preventing or otherwise inhibiting bunching or other deformation of the expandable passage member 408 during implantation of the vascular introducer system 400 within a target vessel. The inner tubular member 410 can extend from a proximal end 426, which can be configured to be positioned proximal to the other proximal ends 414, 418, to a distal end 428, which can be configured to be positioned distal to the other distal ends 416, 424. As shown, the distal end 428 of the inner tubular member 410 can include a conical-like shape that atraumatically guides the vascular introducer system 400 within the target vessel.

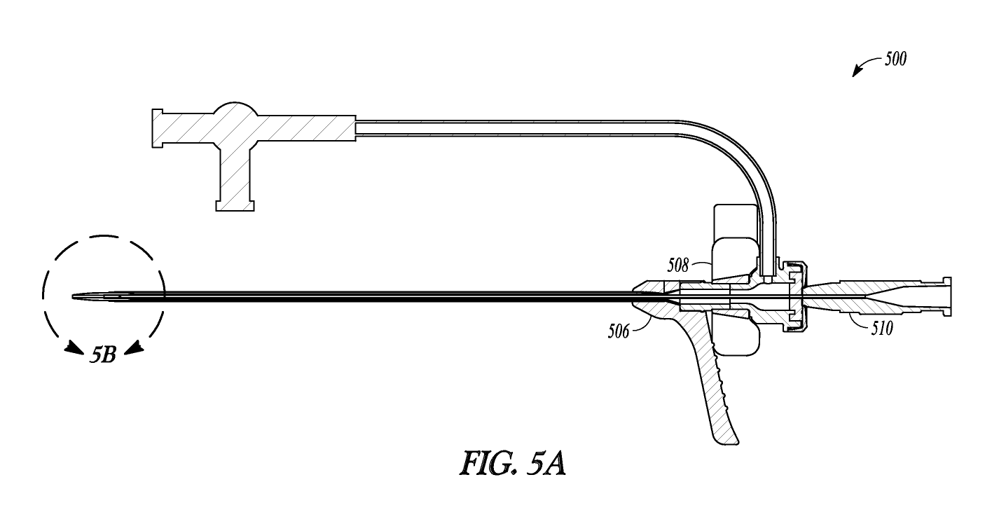

FIG. 5A illustrates a side, cross-sectional view of an example vascular introducer system 500. An expandable passage member 508 of the vascular introducer system 500 is advanced from a skin entry site to a target vessel in a contracted condition. This contracted condition is maintained during implantation by an outer tubular, tear-away member 506 and by an inner tubular member 510. The inner tubular member 510 and outer tubular member 506 can function as barriers for the expandable passage member 508, and can provide column strength to the system 500 as it is inserted within the target vessel.

As illustrated in the enlarged distal portion view of FIG. 5B, the vascular introducer system 500 can be advanced over a guide wire or other “railway” by way of an inner lumen 520 of the inner tubular member 510. This feature can enhance the safety and efficiency with which the vascular introducer system 500 is advanced within the confines of a subject's body. Once the system 500 reaches and is inserted into the target vessel, the inner tubular member 510 and outer tubular member 506 can be easily removed, and the expandable passage member 508 can be expanded. In various examples, to facilitate removal, there is a lack of bonding attachment between the inner tubular member 510 and the expandable passage member 508. Similarly, there is a lack of bonding attachment between the outer tubular, tear-away member 506 and the expandable passage member 508. In lieu of any bonding material being used, the components of the vascular introducer system 500 can be configured to with tight dimensional tolerances and rely on friction fits to avoid premature separation.

FIG. 6 illustrates a transverse cross-sectional view of an example vascular introducer system 600 along its length. This view illustrates a tri-axial configuration of an outer tubular, tear-away member 606, an expandable passage member 608, and an inner tubular member 610. The expandable passage member 608 is configured to receive an elongate treatment device or fluids, such as following removal of both the inner and the outer tubular members 610, 606, via expansion of an introduction channel 624 defined by a wall 622 of the passage member 608. The expandable passage member 608 can include a flexible or flimsy, thin-walled tubular material or sheet. The passage member 608 can be expanded from a contracted condition, as shown in FIG. 6, to an enlarged condition in which the passage member 608 at least partially defines the introduction channel 624, as shown in FIG. 9.

In the contracted or a semi-contracted condition, the expandable passage member 608 can include a folded, wrapped, twisted, rolled or otherwise compressed polymer member extending about a circumference of the inner tubular member 610. The compressed polymer member can be formed using a folding mandrel or vacuum means. In an example, the expandable passage member 608 includes a fold having an overlap amount extending about 360 degrees about the member's axis 670. This overlap decreases in response to a radially-outward directed force on the expandable passage member 608. In an example, the expandable passage member 608 can include one or more non-helical folds along its length and can include a lubricous coating on its inner or outer wall surfaces. In an example, the expandable passage member 608 includes a helically-wrapped sheet of polymer. Each subsequent turn of the polymer sheet can be positioned to partially overlap a previous turn. Other specific constructions are also possible so long as the expandable passage member 608 can assume (a) an initial contracted or collapsed configuration having a sufficiently narrow outer diameter to facilitate vessel penetration of the vascular introducer system 600 and (b) a subsequent expanded configuration after passage of an elongate inner tubular member 610 or treatment device therethrough.

To achieve the expanded or enlarged condition, the expandable passage member 608 can unfold, unwrap, untwist, unroll, or otherwise decompress to at least partially define the introduction channel 624 (e.g., for receiving one or more treatment devices or a fluid, such as a medicament, anti-thrombotic agent, and the like therethrough). The expandable passage member 608 can be configured to expand, as necessary, to accommodate treatment devices of progressively larger profile. In various examples, an inner diameter of the expandable passage member 608 is greater than an inner diameter of the outer tubular, tear-away member 606 post-expansion. In an example, the inner diameter of the expandable passage member 608 is configured to increase in diametrical size by at least 100%, such as from about 4-Fr to about 8-Fr.

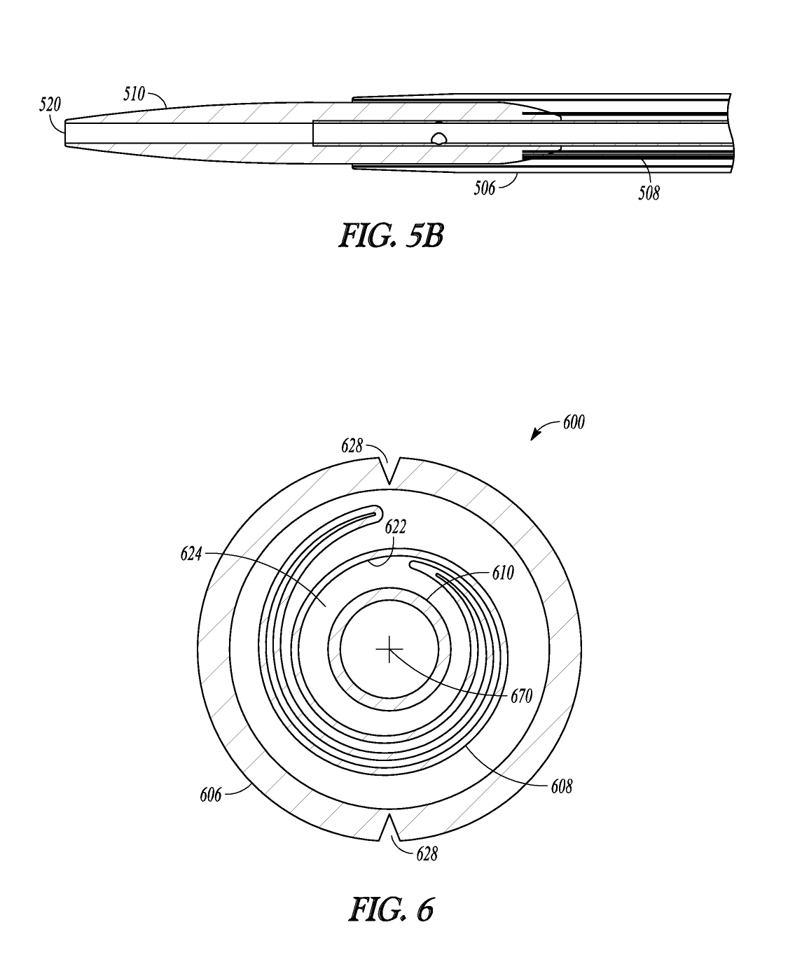

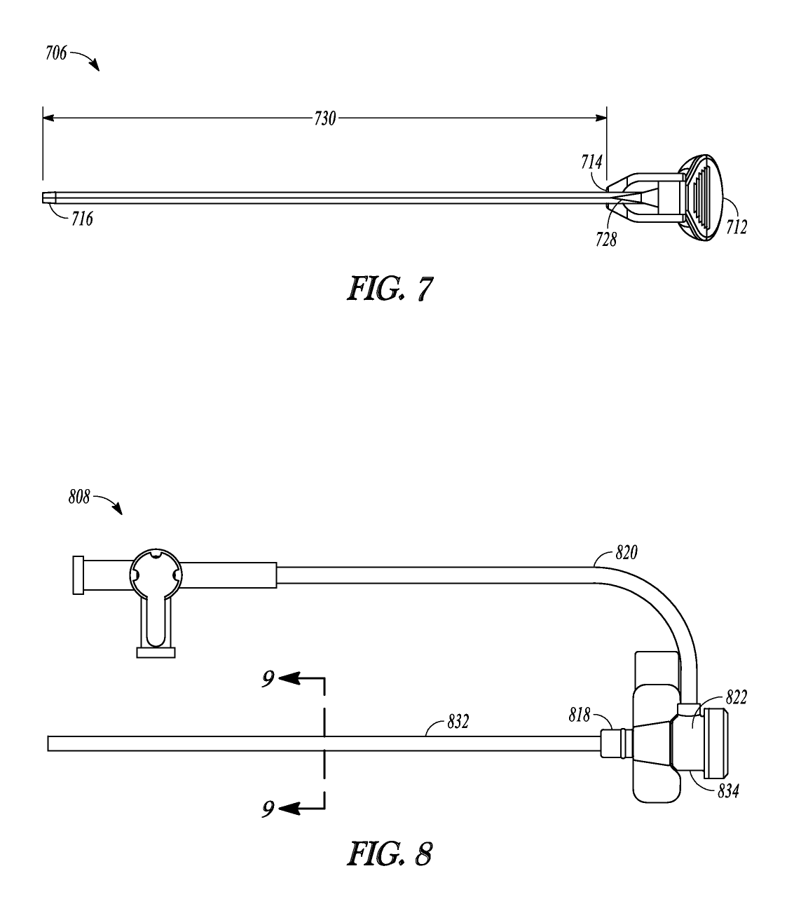

FIG. 7 illustrates a side view of an example removable outer tubular, tear-away member 706 of a vascular introducer system. In some examples, the outer tubular member 706 is formed from a lubricous polymer, such as a polytetrafluoroethylene (PTFE) or fluorinated ethylene propylene (FEP). In order to facilitate removal, the outer tubular member 706 can include a notched split line, score line, linear orientation, or other structure 628 (FIG. 6), 728 allowing separation of the member's material along a portion of its length and can further include a user-engagable peel tab 712 at its proximal end 714.

The outer tubular member 706 can include a slightly tapered distal end 716 to facilitate introduction in a target vessel. Additionally, the exterior surface of the outer tubular member 706 can be wholly or partly coated with a lubricant to further facilitate penetration, although this may not be necessary. In an example, the outer tubular member 706 can include an outer surface diameter of about 6-Fr or less. In an example, the outer tubular member 706 can include an outer surface diameter of about 0.078 inches and an inner surface diameter of about 0.065 inches. In an example, the outer tubular member 706 can include a length 730 of between 4 to 5 inches.

FIG. 8 illustrates a side view of an example expandable passage member 808 of a vascular introducer system. The expandable passage member 808 includes an outer surface 832 configured to contact an inner surface wall of a target vessel following removal of an outer tubular, tear-away member (see, e.g., FIG. 7). A proximal end 818 of the expandable passage member 808 can connect to a side-arm member 820 and can include a hub 834 having a valve member 822. The side-arm member 820 can provide access to an introduction channel 624 (FIG. 6) of the expandable passage member 808. The infusion of fluid into the introduction channel 624 by way of the side-arm member 820 can function to flush the contents of the channel 624. The valve member 822 can allow the introduction channel 624 to be sealed at the proximal end 818 and thus preclude the loss of blood therethrough despite the introduction and removal of treatment devices, of variable outer profiles, through an opening of the valve member 822.

The expandable passage member 808 is formed to be radially expandable, (i.e., expandable from a small initial outside diameter to a larger diameter, which defines the introduction channel 624). The expandable passage member 808 can be deformable or otherwise expandable in the radial direction to permit the desired radial dilation as an inner tubular member or a treatment device is axially advanced therethrough. The expandable passage member 808 can include a lubricous inner surface to facilitate such axial advancement of the inner tubular member or the treatment device, although in some cases it can be sufficient to provide a lubricous outer surface on the inner tubular member or the treatment device itself.

In various examples, as mentioned above, the expandable passage member 808 can include a length that is less than an outer tubular, tear-away member to preserve its integrity during vessel implantation of the vascular introducer system. In an example, the length of the expandable passage member 808 can remain about the same when expanded from a contracted condition, as shown in FIG. 6, to an enlarged condition in which the passage member 808 at least partially defines the introduction channel 924, as shown in FIG. 9. In another example, the length of the expandable passage member 808 decreases when expanded from the contracted condition to the enlarged condition.

FIG. 9 illustrates a transverse cross-sectional view of an example expandable passage member 908 of a vascular introducer system along its length, shown in an enlarged condition defining an introduction channel 924 for delivering treatment devices or fluids into a target vessel. The expandable passage member 908 can be advanced from a skin entry site to the target vessel in a contracted condition. Once the expandable passage member 908 reaches the target vessel lumen, the expandable passage member 908 can be expanded to the enlarged condition, thereby defining the introduction channel 924 within the passage wall 922, and treatment devices or fluids can be introduced into the vessel lumen to perform a minimally invasive procedure. Upon completing the procedure, the expandable passage member 908 can be removed from the vessel.

In various examples, the expandable passage member 908 includes a relatively thin passage wall 922 having low column or axial strength. In an example, the passage wall 922 includes a thickness of about 0.001 inches to 0.002 inches, such as about 0.0015 inches. Because the passage wall 922 is relatively thin-walled, the vascular introducer system—including the expandable passage member 908, an outer tubular member, and an inner tubular member—can attain a relatively low profile (e.g., less than about 6-Fr) when the expandable passage member 908 is in its contracted condition. The thin-walled nature of the passage wall 922 provides little resistance to expansion or contraction, and can conform substantially to vessel anatomy within which it is deployed. The passage wall 922 is not biased to assume any particular configuration or shape upon expansion, and therefore, can adopt whatever shape or configuration that is imposed upon it (e.g., by being folder or otherwise compressed, or by being subjected to internal pressure or force).

The passage wall 922 of the expandable passage member 908 can be constructed of a variety of low-resistant polymer materials that may be fabricated to a relatively thin, flexible configuration (e.g., PTFE, expanded PTFE, FEP, polyethylene teraphathalate (PET), urethane, olefins, polyethylene (PE), silicone, latex, isoprene, chronoprene, and the like). The passage wall 922 can be formed from a lubricious material or hydrophilically coated with a liquid silicone or other coating for facilitating inserting one or more treatment devices (not shown) through the introduction channel 924. In various examples, the passage wall 922 is formed from substantially inelastic material. Alternatively, the passage wall 922 can be formed from an elastic material.

The expandable passage member 908 can include one or more kink-resistant members 950, which extend along a portion of the passage member's 908 length. The kink-resistant members 950 can help inhibit the expandable passage member 908 from assuming a configuration including sharp angles or buckling, such as may be encountered upon pushing an end of the member 908, by increasing its column strength. The kink-resistant members 950 can be constructed from a wire, thread, or filament made of metal, plastic, or a composite material. By way of example, a kink-resistant member 950 can include a configuration made of ground NiTi wire having an outer diameter of about 0.010 inches or less, such as about 0.004 inches. In an example, the kink-resistant members 950 extend along one side of the expandable passage member 908. In an example, the kink-resistant members 950 extend helically around a portion of the expandable passage member 908. Additionally or alternatively, the kink-resistant members 950 can be embedded within, or attached to, a thickened wall region of the expandable passage member 908. Additionally or alternatively, the expandable passage member 908 can include an inner passage member and an outer passage member, and the kink-resistant members 950 can be positioned between a wall of the inner passage member and a wall of the outer passage member.

FIG. 10 illustrates a side view of an example removable inner tubular member 1010 of a vascular introducer system. The inner tubular member 1010 can provide increased column strength to the vascular introducer system during implantation within a subject's body and can provide an atraumatic leading edge portion. The inner tubular member 1010 can be inserted down the entire length of an expandable passage member and result in uniform radial expansion of the passage member for subsequent receipt of a desired treatment device.

The inner tubular member 1010 can include a luer hub 1052 at its proximal end 1026 and can include a dilator 1054 at its distal end 1028. The luer hub 1052 can be coupled to syringes and other peripheral devices. In an example, the working length 1056 between the luer hub 1052 and the dilator 1054 can be about 5.5 inches. Throughout the luer hub 1052, the working length 1056, and the dilator 1054, an inner lumen extends and is configured to receive a guide wire, which can be used throughout a minimally invasive medical procedure.

### Closing Notes

Vessel cross-sectional access size constitutes one of the principal limitations of minimally invasive medical procedures. Advantageously, the present vascular introducer systems, kits, and methods preserve vessel cross-sectional access size and allow diagnostic, therapeutic, and other treatment devices to be inserted into a radial or femoral artery as desired by a caregiver. These treatment devices can include an outer surface diameter approximately equal to, or in some cases greater than, the natural inner diameter of a vessel. At the same, the present systems, kits, and methods provide a low-resistance member, in the form of an expandable passage member, which can be placed between the outer surface of the treatment device and the inner surface of a vessel wall to inhibit direct treatment device-vessel contact. In various examples, the expandable passage member can be configured to be inserted at a first, smaller diameter and be expanded to a second, larger diameter after being positioned in a target vessel. The second, larger diameter can define an introduction channel for receiving the treatment devices.

Among other things, it is believed that the expandable passage member can: (a) reduce axial stress on a vessel and associated pain or discomfort experienced by a subject, (b) inhibit involuntary vessel spasm, and (c) protect vessel walls as a treatment device is introduced into a vessel, (d) without compromising vessel access size to an appreciable degree. Additionally, the expandable passage member can accommodate natural vessel geometry and characteristics in terms of vessel dimensions, ductility, and operability due to its low column strength and thin-walled configuration.

The above Detailed Description includes references to the accompanying drawings, which form a part of the Detailed Description. The drawings show, by way of illustration, specific embodiments in which the present vascular introducer systems, kits, and methods can be practiced. These embodiments are also referred to herein as “examples.”

The above Detailed Description is intended to be illustrative, and not restrictive. For example, the above-described examples (or one or more elements thereof) can be used in combination with each other. Other embodiments can be used, such as by one of ordinary skill in the art upon reviewing the above description. Also, various features or elements can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

In this document, the terms “a” or “an” are used to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “about” and “approximately” are used to refer to an amount that is nearly, almost, or in the vicinity of being equal to a stated amount. In this document, the terms “proximal” and “distal” are used to refer to a system element location relative to a caregiver user. For example, a proximal element portion is a portion closer to the user of the system, whereas a distal element portion is a portion farther away from the user of the system, such as the portions interacting with a subject recipient. In this document, the term “subject” is meant to include mammals, such as for human applications or veterinary applications. Finally, in this document, the term “tear-away” is intended to include removal of a member by splitting, peeling, cutting and the like along a split, score line, linear orientation, or other structure allowing longitudinal separation of the member's material.

In the appended claims, the terms “including” and “in which” are used as the plain-English equivalents of the respective terms “comprising” and “wherein.” Also, in the following claims, the terms “including” and “comprising” are open-ended, that is, a system, kit, or method that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The Abstract is provided to allow the reader to quickly ascertain the nature of the technical disclosure. It is submitted with the understanding that it will not be used to interpret or limit the scope or meaning of the claims.

What is claimed is:

1. A vascular introducer system, comprising:
a removable inner tubular member;
a removable outer tubular, tear-away member; and
an expandable passage member positioned between the inner tubular member and the outer tubular, tear-away member, the expandable passage member configured to sufficiently allow for controlled inelastic radial expansion upon application of a radial force thereto,
wherein the expandable passage member includes an inner surface configured to receive an elongate treatment device following removal of both the inner and the outer tubular members.

2. The vascular introducer system of claim 1, wherein the expandable passage member includes a folded, wrapped, or rolled polymer member extending about a circumference of the inner tubular member.

3. The vascular introducer system of claim 1, wherein the expandable passage member includes a wall thickness of about 0.001 inches to about 0.002 inches.

4. The vascular introducer system of claim 1, wherein an inner diameter of the outer tubular, tear-away member is less than an inner diameter of the expandable passage member, post-expansion.

5. The vascular introducer system of claim 1, wherein the outer tubular, tear-away member includes an outer surface diameter of about 6-Fr or less.

6. The vascular introducer system of claim 1, wherein an initial configuration of the expandable passage member is maintained during implantation within a vessel by the outer tubular, tear-away member and by the inner tubular member.

7. The vascular introducer system of claim 1, wherein the expandable passage member includes one or more kink-resistant members extending along a length portion of the expandable passage member.

8. The vascular introducer system of claim 7, wherein the one or more kink-resistant members extend along one side of the expandable passage member.

9. The vascular introducer system of claim 7, wherein the one or more kink-resistant members include a wire configuration having an outer diameter of about 0.004 inches or less.

10. The vascular introducer system of claim 7, wherein the one or more kink-resistant members are embedded within, or attached to, a wall of the expandable passage member.

11. The vascular introducer system of claim 7, wherein the expandable passage member includes an inner passage member and an outer passage member, and wherein the one or more kink-resistant members are positioned between a wall of the inner passage member and a wall of the outer passage member.

12. The vascular introducer system of claim 1, wherein there is a lack of bonding attachment between the inner tubular member and the expandable passage member and between the outer tubular, tear-away member and the expandable passage member.

13. The vascular introducer system of claim 1, wherein the expandable passage member includes an outer surface configured to contact an inner surface of a vessel following removal of the outer tubular, tear-away member.

14. A kit, comprising:
a needle;
a guide wire;
the vascular introducer system of claim 1; and
instructions for using the vascular introducer system to insert an elongate treatment device into a radial or a femoral artery.

15. A method comprising:
inserting at least a portion of an inner tubular member, an expandable passage member, and an outer tubular member into a vessel;
separating and removing the outer tubular member;
removing the inner tubular member; and
radially expanding an inner surface of the expandable passage member from a first diametrical size to a larger, second diametrical size, including introducing a radial force against the inner surface sufficient to inelastically expand the expandable passage member.

16. The method of claim 15, wherein introducing the radial force includes inserting an elongate treatment device, having an outer diameter greater than an outer diameter of the inner tubular member, into the expandable passage member thereby radially expanding the expandable passage member from a proximal end portion to a distal end portion.

17. The method of claim 16, wherein introducing the elongate treatment device into the expandable passage member includes protecting the vessel by inhibiting direct contact between an inner vessel surface and an outer surface of the elongate treatment device.

18. The method of claim 15, wherein introducing the radial force includes introducing an elongate treatment device, having an outer diameter equal to or greater than about 6-Fr, into a radial artery such that a wall of the expandable passage member is positioned intermediate an outer surface of the treatment device and an inner surface of the radial artery.

19. The method of claim 15, wherein introducing the radial force includes introducing an elongate treatment device, having an outer diameter equal to or greater than about 9-Fr, into a femoral artery such that a wall of the expandable passage member is positioned intermediate an outer surface of the treatment device and an inner surface of the femoral artery.

20. The method of claim 15, wherein inserting the inner tubular member, the expandable passage member, and the outer tubular member into the vessel includes guiding an inner lumen of the inner tubular member over a guide wire.

21. The method of claim 15, wherein inserting the inner tubular member, the expandable passage member, and the outer tubular member into the vessel includes inserting the members into a radial artery.

22. The method of claim 15, wherein radially expanding the inner surface of the expandable passage member includes increasing a diametrical size of the inner surface by at least about 100%.
